(19) United States
(12) Patent Application Publication
(10) Pub. No.: US 2009/0317807 A1
(43) Pub. Date: Dec. 24, 2009

(54) Primers for Use in Detecting Metallo-Beta-Lactamases

(75) Inventor: Nancy D. Hanson, Gretna, NE (US)

Correspondence Address: MUETING, RAASCH & GEBHARDT, P.A., P.O. BOX 581336, MINNEAPOLIS, MN, 55458-1336, US

(73) Assignee: Creighton University, Omaha, NE (US)

(21) Appl. No.: 12/225,841

(22) PCT Filed: May 25, 2007
(86) PCT No.: PCT/US2007/012629

§ 371 Date: Dec. 15, 2008

Related U.S. Application Data
(60) Provisional application No. 60/808,441, filed on May 25, 2006.

(51) Int. Cl. C12Q 1/68; C07H 21/04

(52) U.S. Cl. 435/6; 536/24.33

(57) ABSTRACT

Oliognucleotide primers are provided that are specific for nucleic acid characteristic of certain beta-lactamases. The primers can be employed in methods to identify nucleic acid characteristic of family-specific beta-lactamase enzymes in samples, and particularly, in clinical isolates of Gram-negative bacteria.

### STATEMENT OF RELATED APPLICATIONS

The present application claims priority to U.S. Provisional Patent Application Ser. No. 60/808,441, filed on May 25, 2006, which is incorporated herein by reference in its entirety.

### BACKGROUND

A disturbing consequence of the use, and over-use, of beta-lactam antibiotics (e.g., penicillins, cephalosporins, and carbapenems) has been the development and spread of new beta-lactamases. Beta-lactamases are enzymes that open the beta-lactam ring of penicillins, cephalosporins, carbapenems, and related compounds, to inactivate the antibiotic. The production of beta-lactamases is an important mechanism of resistance to beta-lactam antibiotics among Gram-negative bacteria.

Expanded-spectrum cephalosporins have been specifically designed to resist degradation by the older broad-spectrum beta-lactamases such as TEM-1, 2, and SHV-1. Microbial response to the expanded-spectrum cephalosporins has been the production of mutant forms of the older beta-lactamases called extended-spectrum beta-lactamases (ESBLs). Although ESBL-producing Enterobacteriaceae were first reported in Europe in 1983 and 1984, ESBLs have now been found in organisms of diverse genera recovered from patients in all continents except Antarctica. The occurrence of ESBL-producing organisms varies widely with some types more prevalent in Europe (TEM-3), others more prevalent in the United States (TEM-10, TEM-12 and TEM-26), while others appear worldwide (SHV-2 and SHV-5). These enzymes are capable of hydrolyzing the newer cephalosporins and aztreonam. Studies with biochemical and molecular techniques indicate that many ESBLs are derivatives of older TEM-1, TEM-2, or SHV-1 beta-lactamases, some differing from the parent enzyme by one or more acid substitutions.

Furthermore, carbapenem-hydrolyzing beta-lactamases are some of the more recently described beta-lactamases in the repertoire of penicillin-interactive proteins. These enzymes are responsible for conferring resistance to the carbapenems, the -lactam class with the broadest spectrum of antibacterial activity, by preferentially hydrolyzing carbapenems (Rasmussen et al., Antimicrobial Agents and Chemotherapy, 41(2):223-232 (Feb. 1997)). On a molecular level these enzymes can belong either to the class A group of beta-lactamases that have serine at the active site or to the class B enzymes that represent only the metallo-beta-lactamases identified (Ambler, Philos. Trans. R. Soc. London Biol., 289:321-331 (1980)).

Metallo-beta-lactamases hydrolyze most beta-lactam antibiotics except aztreonam. Therefore, many pathogens that produce these enzymes at high levels are resistant to the majority of beta-lactam antibiotics, including oxyiminocephalosporins and carbapenems. Metallo-beta-lactamases are unique as compared with serine beta-lactamases because these enzymes require the metal ion zinc for substrate hydrolysis (Cricco et al., Current Pharm. Design, 5:915-927 (1999)).

The first report of an imported metallo-beta-lactamase described a Pseudomonas aeruginosa isolate obtained from a Japanese patient in 1988 (Watanabe et al., Antimicrob. Agents Chemother., 35:147-151 (1991)). Since then, the occurrence of mobile genetic elements encoding metallo-beta-lactamases has extended beyond P. aeruginosa to include many types of Gram-negative organisms (e.g., Serratia marcescens, E. coli, Klebsiella pneumoniae, and Acinetobacter spp.) distributed throughout the world (Walsh et al., Clin. Microbiol. Rev., 18:306-325 (2005)). Areas which have reported these types of isolates include several countries in Asia and Europe; the Americas, including Brazil, Canada, and the United States; and Australia (Walsh et al., Clin. Microbiol. Rev., 18:306-325 (2005)). These data are disturbing because acquisition of metallo-beta-lactamases by, e.g., P. aeruginosa reduces the few available options for therapy for infections caused by this pathogen. In addition, the mobility of the genetic elements from which metallo-beta-lactamases are expressed increases the ability of this resistance mechanism to spread to other Gram-negative pathogens (Walsh et al., Clin. Microbiol. Rev., 18:306-325 (2005)).

It is of particular concern that genes encoding the beta-lactamases are often located on large plasmids that also contain genes for resistance to other antibiotic classes including aminoglycosides, tetracycline, sulfonamides, trimethoprim, and chloramphenicol. Furthermore, there is an increasing tendency for pathogens to produce multiple beta-lactamases. These developments, which occur over a wide range of Gram-negative genera, represent a recent evolutionary development in which common Gram-negative pathogens are availing themselves of increasingly complex repertoires of antibiotic resistance mechanisms. Clinically, this increases the difficulty of identifying effective therapies for infected patients.

Thus, there is a need for techniques that can quickly and accurately identify the types of beta-lactamases that may be present in a clinical isolate or sample, for example. This could have significant implications in the choice of antibiotic necessary to treat a bacterial infection.

### SUMMARY OF THE INVENTION

The present invention is directed to the use of oligonucleotide primers specific to nucleic acids characteristic of (typically, genes encoding) certain beta-lactamase enzymes. More specifically, the present invention uses primers to identify family specific beta-lactamase nucleic acids (typically, genes) in samples, particularly, in clinical isolates of Gram-negative bacteria. Specific primers of the invention include the primer sequences set forth in SEQ ID NOs: 1-10. As used herein, a nucleic acid characteristic of a beta-lactamase enzyme includes a gene or a portion thereof. A “gene” as used herein, is a segment or fragment of nucleic acid (e.g., a DNA molecule) involved in producing a peptide (e.g., a polypeptide and/or protein). A gene can include regions preceding (upstream) and following (downstream) a coding region (i.e., regulatory elements) as well as intervening sequences (introns, e.g., non-coding regions) between individual coding segments (exons). The term “coding region” is used broadly herein to mean a region capable of being transcribed to form an RNA, the transcribed RNA can be, but need not necessarily be, further processed to yield an mRNA.

Additionally, a method for identifying a beta-lactamase in a clinical sample is provided. Preferably, the clinical sample provided is characterized as a Gram-negative bacteria with resistance to beta-lactam antibiotics. The method includes, providing a pair of oligonucleotide primers, wherein one primer of the pair is complementary to at least a portion of the beta-lactamase nucleic acid in the sense strand and the other primer of each pair is complementary to a different portion of the beta-lactamase nucleic acid in the antisense strand; annealing the primers to the beta-lactamase nucleic acid; simultaneously extending the annealed primers from a 3′ terminus of each primer to synthesize an extension product complementary to the strands annealed to each primer wherein each extension product after separation from the beta-lactamase nucleic acid serves as a template for the synthesis of an extension product for the other primer of each pair; separating the amplified products; and analyzing the separated amplified products for a size characteristic of the type of beta-lactamase gene.

The method, described above, can employ oligonucleotide primers that are specific for nucleic acid of the IMP family of metallo-beta-lactamases and the VIM family of metallo-beta-lactamases. Primers that can be used include those that are specific for nucleic acid of the metallo-beta-lactamases originating from Pseudomonas aeruginosa, Serratia marcescens, E. coli, Klebsiella pneumoniae, Acinetobacter spp., Enterobacter cloacae, Citrobacter freundii, Morganella morganii, Hafnia alvei, or any other Gram-negative organism capable of accepting foreign DNA.

Oligonucleotide primers that are suitable for use in the present invention include primers that are specific for nucleic acid of the metallo-beta-lactamases of the IMP-family, for example, those designated as IMP-1, IMP-2, IMP-4, IMP-5, IMP-6, IMP-7, IMP-8, IMP-9, IMP-10, IMP-11, IMP-13, IMP-18, IMP-19, and IMP-21. Other oligonucleotide primers that are suitable for use in the present invention include primers that are specific for nucleic acid of the VIM family of metallo-beta-lactamases. It is currently believed that the VIM family of metallo-beta-lactamases are highly related, and that the primers of the present invention that are specific for VIM-type metallo-beta-lactamases will identify most, if not all, VIM genes. VIM-family beta-lactamases currently known in the art include those designated as VIM-1 through VIM-10, VIM-11A, and VIM-11B.

The present invention is further directed to diagnostic kits for detecting and/or identifying beta-lactamases, such as metallo-beta-lactamases, in a sample. The detection and/or identification may be performed by, e.g., polymerase chain reaction (PCR). Such kits may include packaging, containing, separately packaged, at least one primer capable of hybridizing to beta-lactamase nucleic acid of interest, for instance, the IMP family of beta-lactamases and the VIM family of beta-lactamases; a positive and negative control, and a protocol for identification of the beta-lactamase nucleic acid of interest. The primers may be selected from the group consisting of SEQ ID NOs:1-10.

### BRIEF DESCRIPTION OF THE DRAWINGS

FIG. 1 provides the primers of the present invention.

### DETAILED DESCRIPTION OF CERTAIN EMBODIMENTS

The present invention is directed to the detection of nucleic acid that is characteristic of (e.g., at least a segment of a gene that codes for) family-specific beta-lactamase nucleic acid in samples (e.g., clinical isolates of Gram-negative bacteria). Specifically, the present invention is directed to the detection of beta-lactamase nucleic acid (preferably, a gene or at least a segment of a gene) using unique primers and the polymerase chain reaction. Using the primers and methods of the present invention, metallo-beta-lactamases belonging to the IMP family and the VIM family, for example, can be identified.

The primers and methods of the present invention are useful for a variety of purposes, including, for example, the identification of the primary beta-lactamase(s) responsible for resistance to third generation cephalosporins, in addition to the carbapenems, among any Gram-negative organism capable of accepting foreign DNA. Such Gram-negative bacteria include, but are not limited to, Pseudomonas aeruginosa (Walsh et al., Clin. Microbiol. Reviews, 18(2):306-325 (2005); Patrice et al., Antimicrob. Agents Chemother., 37(5):962-969 (1993)); as well as Escherichia coli and Klebsiella pneumoniae (Thomson et al., Antimicrob. Agents Chemother., 36(9):1877-1882 (1992)). Other sources of beta-lactamases include, for example, a wide range of Enterobacteriaceae, including Enterobacter spp., Citrobacter freundii, Morganella morganii, Providencia spp., and Serratia marcescens (Jones, Diag. Microbiol. Infect. Disease., 31(3):461-466 (1998)). Additional beta-lactamase gene sources include Proteus mirabilis (Bret et al., Antimicrob. Agents Chemother., 42(5):1110-1114 (1998)); Yersinia enterocolitica (Barnaud et al., FEMS Microbiol. Letters, 148(1):15-20 (1997)); and Klebsiella oxytoca (Marchese et al., Antimicrob. Agents Chemother., 42(2):464-467 (1998)).

The methods of the present invention involve the use of the polymerase chain reaction sequence amplification method (PCR) using novel primers. U.S. Pat. No. 4,683,195 (Mullis et al.) describes a process for amplifying, detecting, and/or cloning nucleic acid. Preferably, this amplification method relates to the treatment of a sample containing nucleic acid (typically, DNA) of interest from bacteria, particularly Gram-negative bacteria, with a molar excess of an oligonucleotide primer pair, heating the sample containing the nucleic acid of interest to yield two single-stranded complementary nucleic acid strands, adding the primer pair to the sample containing the nucleic acid strands, allowing each primer to anneal to a particular strand under appropriate temperature conditions that permit hybridization, providing a molar excess of nucleotide triphosphates and polymerase to extend each primer to form a complementary extension product that can be employed in amplification of a desired nucleic acid, detecting the amplified nucleic acid, and analyzing the amplified nucleic acid for a size specific amplicon (as indicated below) characteristic of the specific beta-lactamase of interest. This process of heating, annealing, and synthesizing is repeated many times, and with each cycle the desired nucleic acid increases in abundance. Within a short period of time, it is possible to obtain a specific nucleic acid, e.g., a DNA molecule, that can be readily purified and identified.

The oligonucleotide primer pair includes one primer that is substantially complementary to at least a portion of a sense strand of the nucleic acid and one primer that is substantially complementary to at least a portion of an antisense strand of the nucleic acid. As used herein, substantially complementary refers to primers wherein changes in one or more nucleotide bases do not affect their ability to hybridize to the target nucleic acid. The process of forming extension products preferably involves simultaneously extending the annealed primers from a 3′ terminus of each primer to synthesize an extension product that is complementary to the nucleic acid strands to which each primer is annealed, wherein each extension product after separation from the beta-lactamase nucleic acid includes a sequence complementary to the other primer. The amplified products are preferably detected by size fractionization using gel electrophoresis. Variations of the method are described in U.S. Pat. No. 4,683,194 (Saiki et al.). The polymerase chain reaction sequence amplification method is also described by Saiki et al., Science, 230, 1350-1354 (1985) and Saiki et al., Nature, 324, 163-166 (1986).

An “oligonucleotide,” as used herein, refers to a polymeric form of nucleotides of any length, either ribonucleotides or deoxyribonucleotides. The term oligonucleotide refers particularly to the primary structure, and thus includes double and single-stranded DNA molecules and double and single-stranded RNA molecules.

A “primer,” as used herein, is an oligonucleotide that is complementary to at least a portion of nucleic acid of interest and, after hybridization to the nucleic acid, may serve as a starting-point for the polymerase chain reaction. The terms “primer” or “oligonucleotide primer,” as used herein, further refer to a primer, having a nucleotide sequence that possesses a high degree of nucleic acid sequence similarity to at least a portion of the nucleic acid of interest. “High degree” of sequence similarity refers to a primer that typically has at least about 80% nucleic acid sequence similarity, and preferably at least about 90% nucleic acid sequence similarity. Sequence similarity may be determined, for example, using sequence techniques such as GCG FastA (Genetics Computer Group, Madison, Wis.), MacVector 4.5 (Kodak/IBI software package) or other suitable sequencing programs or methods known in the art.

The terms “complement” and “complementary” as used herein, refer to a nucleic acid that is capable of hybridizing to a specified nucleic acid molecule under stringent hybridization conditions. Stringent hybridization conditions include, for example, temperatures from about 50 degrees Celsius (° C.) to about 65° C., and magnesium chloride (MgCl₂) concentrations from about 1.5 millimolar (mM) to about 2.0 mM. Thus, a specified DNA molecule is typically “complementary” to a nucleic acid if hybridization occurs between the specified DNA molecule and the nucleic acid. Under certain conditions, hybridization may occur even if the sequences of the DNA molecule and the nucleic acid are not perfectly complementary but are mismatched at one or more positions. If the specified DNA molecule hybridizes to the full-length of the nucleic acid molecule, then the specified DNA molecule is typically a “full-length complement.” “Complementary” further refers to the capacity of purine and pyrimidine nucleotides to associate through hydrogen bonding in double stranded nucleic acid molecules. The following base pairs are complementary: guanine and cytosine; adenine and thymine; and adenine and uracil.

As used herein, the terms “amplified molecule,” “amplified fragment,” and “amplicon” refer to a nucleic acid molecule (typically, DNA) that is a copy of at least a portion of the nucleic acid and its complementary sequence. The copies correspond in nucleotide sequence to the original molecule and its complementary sequence. The amplicon can be detected and analyzed by a wide variety of methods. These include, for example, gel electrophoresis, single strand conformational polymorphism (SSCP), restriction fragment length polymorphism (RFLP), capillary zone electrophoresis (CZE), and the like. Preferably, the amplicon can be detected, and hence, the type of beta-lactamase identified, using gel electrophoresis and appropriately sized markers, according to techniques known to one of skill in the art.

The primers are oligonucleotides, either synthetic or naturally occurring, capable of acting as a point of initiating synthesis of a product complementary to the region of the DNA molecule containing the beta-lactamase of interest. The primer includes nucleotides capable of hybridizing under stringent conditions to at least a portion of at least one strand of a nucleic acid molecule of a given beta-lactamase. Preferably, the primers of the present invention typically have at least about 12 nucleotides and more preferably at least about 15 nucleotides. Preferably, the primers have no more than about 35 nucleotides, and more preferably, no more than about 25 nucleotides. The primers are chosen such that they preferably produce a primed product of about 50-1300 base pairs.

Optionally, a primer used in accordance with the present invention includes a label constituent. The label constituent can be selected from the group of an isotopic label, a fluorescent label, a polypeptide label, and a dye release compound. The label constituent is typically incorporated in the primer by including a nucleotide having the label attached thereto. Isotopic labels preferably include those compounds that are beta, gamma, or alpha emitters, more preferably isotopic labels are selected from the group of ³²P, ³⁵S, and ¹²⁵I. Fluorescent labels are typically dye compounds that emit visible radiation in passing from a higher to a lower electronic state, typically in which the time interval between adsorption and emission of energy is relatively short, generally on the order of about 10⁻⁸ to about 10⁻³ second. Suitable fluorescent compounds that can be utilized include fluorescien and rhodamine, for example. Suitable polypeptide labels that can be utilized in accordance with the present invention include antigens (e.g., biotin, digoxigenin, and the like) and enzymes (e.g., horse radish peroxidase). A dye release compound typically includes chemiluminescent systems defined as the emission of absorbed energy (typically as light) due to a chemical reaction of the components of the system, including oxyluminescence in which light is produced by chemical reactions involving oxygen.

Preferred examples of these primers that are specific for certain beta-lactamases are as follows, wherein “F” in the designations of the primers refers to a 5′ upstream primer and “R” refers to a 3′ downstream primer. For those beta-lactamases that have more than one upstream primer and more than one downstream primer listed below as preferred primers, various combinations can be used. Typically, hybridization conditions utilizing primers of the invention include, for example, a hybridization temperature of about 40° C. to about 68° C., and a MgCl₂ concentration of about 1.5 mM to about 2.0 mM. Although lower or higher temperatures and higher concentrations of MgCl₂ can be employed, this may result in decreased primer specificity.

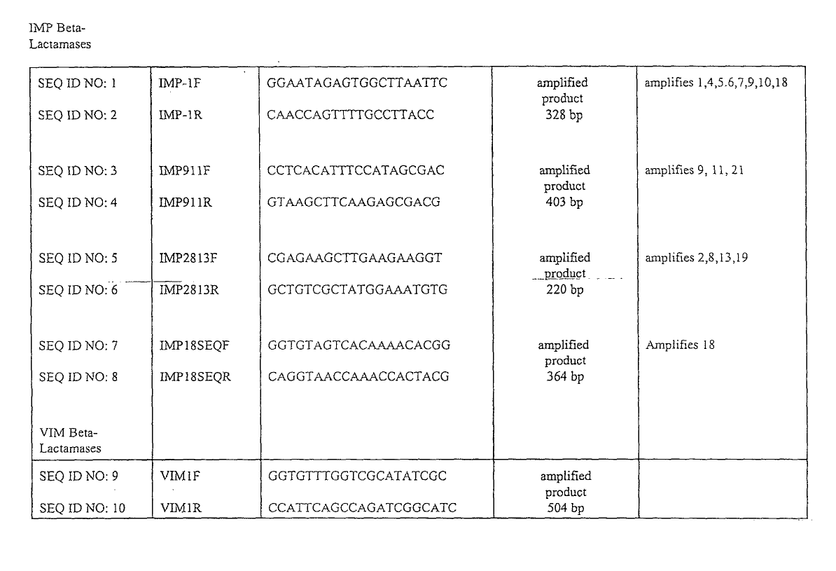

The following primers are specific for nucleic acid characteristic of the IMP-family and VIM-family, that is the IMP-type and the VIM-type, beta-lactamase enzymes (FIG. 1). It is understood that a “type” of beta-lactamase may differ from another “type” of beta-lactamase in some manner. However, despite any such difference, both “types” of beta-lactamase typically will be identified using the same primers. For example, VIM-11A (a VIM-11-type beta-lactamase) differs from VIM-11B (another VIM-11-type beta-lactamase) by a single mutation, but the primers specific for nucleic acid characteristic of VIM-11A beta-lactamase may also be specific for nucleic acid characteristic of VIM-11B beta-lactamase (e.g., primers that identify the VIM-type beta-lactamase VIM-11A will also identify the VIM-type beta-lactamase VIM-11B).

The following primers are specific for nucleic acid characteristic of the IMP-1-type, IMP-4-type, IMP-5-type, IMP-6-type, IMP-7-type, IMP-9-type, IMP-10-type, and IMP-18-type beta-lactamase enzymes.

| | |
|---|---|
| Primer Name: | IMP-1F |
| Primer Sequence: | 5′-GGAATAGAGTGGCTTAATTC-3′ |
| | (SEQ ID NO: 1) |
| Primer Name: | IMP-1R |
| Primer Sequence: | 5′-CAACCAGTTTTGCCTTACC-3′ |
| | (SEQ ID NO: 2) |

Employing a primer pair containing the primer sequences of SEQ ID NO:1 and SEQ ID NO:2 to a sample known to contain any of IMP-1-type, IMP-4-type, IMP-5-type, IMP-6-type, IMP-7-type, IMP-9-type, IMP-10-type, and/or IMP-18-type beta-lactamases, a size-specific amplicon of 328 base pairs will typically be obtained.

The following primers are specific for nucleic acid characteristic of the IMP-9-type, IMP-11-type, and IMP-21-type beta-lactamase enzymes.

| | |
|---|---|
| Primer Name: | IMP911F |
| Primer Sequence: | 5′-CCTCACATTTCCATAGCGAC-3′ |
| | (SEQ ID NO: 3) |
| Primer Name: | IMP911R |
| Primer Sequence: | 5′-GTAAGCTTCAAGAGCGACG-3′ |
| | (SEQ ID NO: 4) |

Employing a primer pair containing the primer sequences of SEQ ID NO:3 and SEQ ID NO:4 to a sample known to contain any of IMP-9-type, IMP-11-type, and/or IMP-21-type beta-lactamases, a size-specific amplicon of 403 base pairs will typically be found.

The following primers are specific for nucleic acid characteristic of the IMP-2-type, IMP-8-type, IMP-13-type, and IMP-19-type beta-lactamase enzymes.

| | |
|---|---|
| Primer Name: | IMP2813F |
| Primer Sequence: | 5′-CGAGAAGCTTGAAGAAGGT-3′ |
| | (SEQ ID NO: 5) |
| Primer Name: | IMP2813R |
| Primer Sequence: | 5′-GCTGTCGCTATGGAAATGTG-3′ |
| | (SEQ ID NO: 6) |

Employing a primer pair containing the primer sequences of SEQ ID NO:5 and SEQ ID NO:6 to a sample known to contain any of IMP-2-type, IMP-8-type, IMP-13-type, and/or IMP-19-type beta-lactamases, a size-specific amplicon of 220 base pairs will typically be obtained.

The following primers are specific for nucleic acid characteristic of the IMP-18-type beta-lactamase.

| | |
|---|---|
| Primer Name: | IMP18SEQF |
| Primer Sequence: | 5′-GGTGTAGTCACAAAACACGG-3′ |
| | (SEQ ID NO: 7) |
| Primer Name: | IMP18SEQR |
| Primer Sequence: | 5′-CAGGTAACCAAACCACTACG-3′ |
| | (SEQ ID NO: 8) |

Employing a primer pair containing the primer sequences of SEQ ID NO:7 and SEQ ID NO:8 to a sample known to contain an IMP-18-type beta-lactamase, a size-specific amplicon of 364 base pairs will typically be found.

It is believed that the above primers (SEQ ID NO:1 through SEQ ID NO:8) will identify a majority (e.g., at least about 83%) of the currently known IMP genes.

The following primers are specific for nucleic acid characteristic of the VIM family of beta-lactamase enzymes.

| | |
|---|---|
| Primer Name: | VIM1F |
| Primer Sequence: | 5′-GGTGTTTGGTCGCATATCGC-3′ |
| | (SEQ ID NO: 9) |
| Primer Name: | VIM1R |
| Primer Sequence: | 5′-CCATTCAGCCAGATCGGCATC-3′ |
| | (SEQ ID NO: 10) |

Employing a primer pair containing the primer sequences of SEQ ID NO:9 and SEQ ID NO:10 to a sample known to contain a VIM-type beta-lactamase, a size-specific amplicon of 504 base pairs will typically be found.

Various other primers, or variations of the primers described above, can also be prepared and used according to methods of the present invention. For example, alternative primers can be designed based on targeted beta-lactamases known or suspected to contain regions possessing high G/C content (i.e., the percentage of guanine and cytosine residues). As used herein, a “high G/C content” in a target nucleic acid, typically includes regions having a percentage of guanine and cytosine residues of about 60% to about 90%. Thus, changes in a prepared primer will alter, for example, the hybridization or annealing temperatures of the primer, the size of the primer employed, and the sequence of the specific resistance gene or nucleic acid to be identified. Therefore, manipulation of the G/C content, e.g., increasing or decreasing, of a primer or primer pair may be beneficial in increasing detection sensitivity in the method.

Additionally, depending on the suspected nucleic acid in the sample, a primer of the invention can be prepared that varies in size. Typically, primers of the invention are about 12 nucleotides to about 35 nucleotides in length, preferably the primers are about 15 nucleotides to about 25 nucleotides in length. Oligonucleotides of the invention can readily be synthesized by techniques known in the art (see, for example, Crea et al., Proc. Natl. Acad. Sci. (U.S.A.), 75:5765 (1978)).

Once the primers are designed, their specificity can be tested using the following method. Depending on the target nucleic acid of clinical interest, nucleic acid template is prepared from a bacterial control strain known to express or contain the resistance gene. This control strain, as used herein, refers to a “positive control” nucleic acid (typically, DNA). Additionally, a “negative control” nucleic acid (typically, DNA) can be isolated from one or more bacterial strains known to express a resistance gene other than the target gene of interest. Using the polymerase chain reaction, the designed primers are employed in a detection method, as described above, and used in the positive and negative control samples and in at least one test sample suspected of containing the resistance gene of interest. The positive and negative controls provide an effective and qualitative (or grossly quantitative) means by which to establish the presence or the absence of the gene of interest of test clinical samples. It should be recognized that with a small percentage of primer pairs, possible cross-reactivity with other beta-lactamase genes might be observed. However, the size and/or intensity of any cross-reactive amplified product will be considerably different and can therefore be readily evaluated and dismissed as a negative result.

The invention also relates to kits for identifying family specific beta-lactamase enzymes by, e.g., PCR analysis. Kits of the invention typically include one or more primer pairs specific for a beta-lactamase of interest, one or more positive controls, one or more negative controls, and protocol for identification of the beta-lactamase of interest using polymerase chain reaction. A negative control includes a nucleic acid (typically, DNA) molecule encoding a resistant beta-lactamase other than the beta-lactamase of interest. The negative control nucleic acid may be a naked nucleic acid (typically, DNA) molecule or be inserted into a bacterial cell. Preferably, the negative control nucleic acid is double stranded; however, a single stranded nucleic acid may be employed. A positive control includes a nucleic acid (typically, DNA) that encodes a beta-lactamase from the family of beta-lactamases of interest. The positive control nucleic acid may be a naked nucleic acid molecule or be inserted into a bacterial cell, for example. Preferably, the positive control nucleic acid is double stranded; however, a single stranded nucleic acid may be employed. Typically, the nucleic acid is obtained from a bacterial lysate.

Accordingly, the present invention provides a kit for characterizing and identifying a family specific beta-lactamase that would have general applicability. Preferably, the kit includes a polymerase (typically, DNA polymerase) enzyme, such as Taq polymerase, and the like. A kit of the invention also preferably includes at least one primer pair that is specific for a beta-lactamase gene or flanking regions of the gene. A buffer system compatible with the polymerase enzyme is also included and is well known in the art. Optionally, at least one primer pair may contain a labeled constituent, a fluorescent label, a polypeptide label, and a dye release compound. The kit may further contain at least one internal sample control, in addition to one or more further means required for, e.g., PCR analysis, such as a reaction vessel. If required, a nucleic acid from the bacterial sample can be isolated and then subjected to PCR analysis using the provided primer set of the invention.

In another embodiment, family specific beta-lactamase enzymes in clinical samples, particularly clinical samples containing Gram-negative bacteria, can be detected by the primers described herein in a “microchip” detection method. In a microchip detection method, nucleic acid, e.g., genes, of multiple beta-lactamases in clinical samples can be detected with a minimal requirement for human intervention. Techniques borrowed from the microelectronics industry are particularly suitable to these ends. For example, micromachining and photolithographic procedures are capable of producing multiple parallel microscopic scale components on a single chip substrate. Materials can be mass produced and reproducibility is exceptional. The microscopic sizes minimize material requirements. Thus, human manipulations can be minimized by designing a microchip type surface capable of immobilizing a plurality of primers of the invention on the microchip surface.

Microchip detection methods generally include the formation of high-density arrays of, for example, oligonucleotides on a surface, typically glass, that can then be used for various applications, such as large scale hybridization (Roth et al., Annu. Rev. Biomed. Eng., 1:265-297 (1999)). Using this method, applications using two types of nucleic acids as targets, synthesizing or printing directly on a surface, or covalent or noncovalent attachment of single stranded cDNA, are known.

According to one of the methods of microchip detection, arrays of short oligonucleotides may be synthesized directly on a surface, such as glass, using methods generally known in solid-phase chemistry synthesis. This is generally accomplished by either masking most of the array, activating the unmasked portion, and adding a phosphoramidite to produce a coupling to the 5′-hydroxy groups of the activated segments of the array or by printing the arrays directly on the surface using ink jet printing techniques.

Alternatively, for production of longer sequences, such as cDNA arrays, a spotting method for attaching molecules to a surface may be used. In this method, for instance, sequences are created from clones, purified, and optionally amplified. The sequences are then attached noncovalently to a glass surface by coating the surface with polylysine or by chemically treating it with an aminosilane to make it cationic. Attachment of the sequences under this method are generally nonspecific and may involve multiple attachment sites along the molecule. An additional method known for attachment of sequences to a surface is to covalently attach amino-modified cDNA, produced by asymmetric PCR, to silylated glass using sodium borohydride (Roth et al., Annu. Rev. Biomed. Eng., 1:265-297 (1999)).

Thus, an object of the present invention is to provide a parallel screening method wherein multiple serial reactions are automatically performed individually within one reaction well for each of the plurality of nucleic acid strands to be detected in the plural parallel sample wells. These serial reactions are performed in a simultaneous run within each of the multiple parallel lanes of the device. “Parallel” as used herein means wells identical in function. “Simultaneous” means within one preprogrammed run. The multiple reactions automatically performed within the same apparatus minimize sample manipulation and labor.

Thus, the present invention provides multiple reaction wells, the reaction wells being reaction chambers, on a microchip, each reaction well containing an individualized array to be used for detecting a beta-lactamase gene uniquely specified by the substrates provided, the reaction conditions and the sequence of reactions in that well. The chip can thus be used as a method for identifying beta-lactamase genes in clinical samples.

Objects and advantages of this invention are further illustrated by the following example, but the particular materials and amounts thereof recited in these examples, as well as other conditions and details, should not be construed to unduly limit this invention.

### Example

### Identification of the IMP-18 Metallo-Beta-Lactamase from a Pseudomonas aeruginosa Isolate

Based on recent reports, there are two major families of imported metallo-beta-lactamases, IMP and VIM, which are carried on mobile gene cassettes inserted into integrons (Walsh et al., Clin. Microbiol. Rev., 18:306-325 (2005)). Including those disclosed herein, there are 18 variants of IMP metallo-beta-lactamases and 11 variants of VIM metallo-beta-lactamases (Watanabe et al., Antimicrob. Agents Chemother., 35:147-151 (1991)), including two published reports on metallo-beta-lactamases from the United States identifying VIM-2 (Lolans et al., Antimicrob. Agents Chemother., 49:3538-3540 (2005)) and VIM 7 (Toleman et al., Antimicrob. Agents Chemother., 48:329-332 (2004)).

The carbapenem-resistant P. aeruginosa isolate investigated in this study was recovered from the tracheal aspirate of a motorcycle accident victim in the southwestern United States. It was found to produce a new member of the IMP family of metallo-beta-lactamases.

The isolate was identified by the Phoenix system (Becton Dickinson Diagnostic Systems, Sparks, Md.). Antibiotic susceptibility was determined by broth microdilution method with investigational dehydrated panels provided by Dade Behring MicroScan (Sacramento, Calif.) according to the manufacturer's recommendations and interpreted according to National Committee for Clinical Laboratory Standards (NCCLS) criteria (National Committee for Clinical Laboratory Standards, “Performance Standards for Antimicrobial Susceptibility Testing, Fourteenth informational supplement 24, Wayne, Pa. (2004)). The data are presented in Table 1.

TABLE 1
Microdilution MICs (μg/ml) of selected antimicrobial
agents against Pseudomonas aeruginosa (IMP-18)

| | Microdilution |
|---|---|
| Antimicrobial Agent | MICs (μg/ml) |
| Imipenem | 64 |
| Ceftazidime | >64 |
| Ceftriaxone | >64 |
| Cefepime | >64 |
| Aztreonam | 32 |
| Ampicillin/Sulbactam | >128 |
| Piperacillin/Tazobactam | 128 |
| Amikacin | 64 |
| Gentamicin | >32 |
| Levofloxacin | >16 |
| Polymyxin B | 4 |
| Minocycline | >128 |
| Tigecycline | 32 |

Metallo-beta-lactamase production was phenotypically investigated by using the Etest metallo-beta-lactamase strip (AB BIODISK, Solna, Sweden). By Etest, there was a ≧32-fold reduction in the MIC of imipenem in the presence of EDTA (≧256 to 8 μg/ml).

The bla_{IMP-1-like} gene was detected by PCR amplification carried out on a DNA Thermal Cycler 480 instrument (Perkin-Elmer, Cetus, Norwalk, Conn.) with the Gene Amp DNA amplification kit containing PlatinumTaq (HIFI) polymerase (Perkin-Elmer, Roche Molecular Systems, Inc., Branchburg, N.J.) using IMP-1-family-specific primers internal to the gene and which flanked the gene. The composition of the reaction mixture was as follows: 10 mM Tris-HCl (pH 8.3), 50 mM KCl, 2 mM MgCl₂, each of the four deoxynucleoside triphosphates at a concentration of 0.2 mM and 1.2 units (U) of PlatinumTaq (HIFI) in a total volume of 49 μl. A total of 1 μl of sample lysate was added to the reaction mixture, the mixture was centrifuged briefly, and 50 μl of mineral oil was layered onto the surface. The PCR program consisted of an initial denaturation step at 96° C. for 15 seconds, followed by 25 cycles of DNA denaturation at 96° C. for 15 seconds, primer annealing at 42° C. for 15 seconds, and primer extension at 72° C. for 2 minutes. Amplification of the full length product of 781 base pairs was obtained by PCR as described above. The forward primer was 5′-GTTAGAAAAGGAAAAGTATG-3′, and the reverse primer was 5′-TGCTGCAACGACTTGTTAG-3′.

PCR products were generated on at least two separate occasions and sequenced by automated cycle sequencing, using an ABI Prism 3100-Avant Genetic analyzer (Applied Biosystems, Foster City, Calif.). Sequence alignments and analyses were performed online using the BLAST program (www.ncbi.nlm.nih.gov). Sequence translation was performed using the DNASIS analysis program (Hitachi Engineering Co., San Francisco, Calif.). The GenBank accession number for the gene is AY780674, and the enzyme has been designated IMP-18.

IMP-8 and IMP-14 were the beta-lactamases most closely related to IMP-18. IMP-18 differed from IMP-14 by 21 amino acids, indicating 91% identity, while IMP-8 differed by 29 amino acids, representing 88% identity. Therefore, IMP-18 represents a distinct variant of this family group.

The complete disclosures of all patents, patent documents, publications, ATCC deposits, electronically available material (e.g., GenBank amino acid and nucleotide sequence submissions), etc., cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

All headings are for convenience of the reader and should not be used to limit the meaning of the text that follows the heading, unless so specified.

### Sequence Listing Free Text

SEQ ID NO:1-10 Primer

1. A primer selected from the group consisting of:

| | |
|---|---|
| 5′-GGAATAGAGTGGCTTAATTC-3′; | (SEQ ID NO: 1) |
| 5′-CAACCAGTTTTGCCTTACC-3′; | (SEQ ID NO: 2) |
| 5′-CCTCACATTTCCATAGCGAC-3′; | (SEQ ID NO: 3) |
| 5′-GTAAGCTTCAAGAGCGACG-3′; | (SEQ ID NO: 4) |
| 5′-CGAGAAGCTTGAAGAAGGT-3′; | (SEQ ID NO: 5) |
| 5′-GCTGTCGCTATGGAAATGTG-3′; | (SEQ ID NO: 6) |
| 5′-GGTGTAGTCACAAAACACGG-3′; | (SEQ ID NO: 7) |
| 5′-CAGGTAACCAAACCACTACG-3′; | (SEQ ID NO: 8 |
| 5′-GGTGTTTGGTCGCATATCGC-3′; | (SEQ ID NO: 9) |
| 5′-CCATTCAGCCAGATCGGCATC-3′; | (SEQ ID NO: 10) |

and full-length complements thereof.

2-5. (canceled)

6. A method for identifying a beta-lactamase nucleic acid in a clinical sample, the method comprising:
providing a clinical sample;
contacting the clinical sample with a pair of oligonucleotide primers specific for nucleic acid characteristic of an IMP-family beta-lactamase enzyme, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase nucleic acid.

7. The method of claim 41 wherein the primers are selected from the group
consisting of:

| | |
|---|---|
| 5′-GGAATAGAGTGGCTTAATTC-3′; | (SEQ ID NO: 1) |
| 5′-CAACCAGTTTTGCCTTACC-3′; | (SEQ ID NO: 2) |

and full-length complements thereof.

8-9. (canceled)

10. The method of claim 42 wherein the primers are selected from the group
consisting of:

| | |
|---|---|
| 5′-CCTCACATTTCCATAGCGAC-3′; | (SEQ ID NO: 3) |
| 5′-GTAAGCTTCAAGAGCGACG-3′; | (SEQ ID NO: 4) |

and full-length complements thereof.

11-28. (canceled)

29. A method for identifying a beta-lactamase nucleic acid in a clinical sample, the method comprising:
providing a clinical sample;
contacting the clinical sample with a pair of oligonucleotide primers specific for nucleic acid characteristic of the VIM-family of beta-lactamase enzymes, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase.

30. The method of claim 29 wherein the primers are selected from the group
consisting of:

| | |
|---|---|
| 5′-GGTGTTTGGTCGCATATCGC-3′; | (SEQ ID NO: 9) |
| 5′-CCATTCAGCCAGATCGGCATC-3′; | (SEQ ID NO: 10) |

and full-length complements thereof.

31. (canceled)

32. A method for identifying a beta-lactamase nucleic acid in a clinical sample, the method comprising:
providing a clinical sample;
contacting the clinical sample with a pair of oligonucleotide primers specific for nucleic acid characteristic of a beta-lactamase enzyme, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase;
wherein the primers are selected from the group consisting of:

| | |
|---|---|
| 5′-GGAATAGAGTGGCTTAATTC-3′; | (SEQ ID NO: 1) |
| 5′-CAACCAGTTTTGCCTTACC-3′; | (SEQ ID NO: 2) |
| 5′-CCTCACATTTCCATAGCGAC-3′; | (SEQ ID NO: 3) |
| 5′-GTAAGCTTCAAGAGCGACG-3′; | (SEQ ID NO: 4) |
| 5′-CGAGAAGCTTGAAGAAGGT-3′; | (SEQ ID NO: 5) |
| 5′-GCTGTCGCTATGGAAATGTG-3′; | (SEQ ID NO: 6) |
| 5′-GGTGTAGTCACAAAACACGG-3′; | (SEQ ID NO: 7) |
| 5′-CAGGTAACCAAACCACTACG-3′; | (SEQ ID NO: 8) |
| 5′-GGTGTTTGGTCGCATATCGC-3′; | (SEQ ID NO: 9) |
| 5′-CCATTCAGCCAGATCGGCATC-3′; | (SEQ ID NO: 10) |

and full-length complements thereof.

33. A diagnostic kit for detecting an IMP family beta-lactamase nucleic acid, wherein the kit comprises:
(a) at least one primer pair capable of hybridizing to a beta-lactamase nucleic acid;
(b) at least one positive control and at least one negative control; and
(c) a protocol for identification of the beta-lactamase nucleic acid, wherein the primers are selected from the group consisting of:

| | |
|---|---|
| 5′-GGAATAGAGTGGCTTAATTC-3′; | (SEQ ID NO: 1) |
| 5′-CAACCAGTTTTGCCTTACC-3′; | (SEQ ID NO: 2) |
| 5′-CCTCACATTTCCATAGCGAC-3′; | (SEQ ID NO: 3) |
| 5′-GTAAGCTTCAAGAGCGACG-3′; | (SEQ ID NO: 4) |
| 5′-CGAGAAGCTTGAAGAAGGT-3′; | (SEQ ID NO: 5) |
| 5′-GCTGTCGCTATGGAAATGTG-3′; | (SEQ ID NO: 6) |
| 5′-GGTGTAGTCACAAAACACGG-3′; | (SEQ ID NO: 7) |
| 5′-CAGGTAACCAAACCACTACG-3′; | (SEQ ID NO: 8) |

and full-length complements thereof.

34-37. (canceled)

38. A diagnostic kit for detecting a VIM family beta-lactamase, wherein the kit comprises:
(a) at least one primer pair capable of hybridizing to a beta-lactamase nucleic acid;
(b) at least one positive control and at least one negative control; and
(c) a protocol for identification of the beta-lactamase nucleic acid, wherein at least one of the primers of the primer pair is selected from the group consisting of:

| | |
|---|---|
| 5′-GGTGTTTGGTCGCATATCGC-3′; | (SEQ ID NO: 9) |
| 5′-CCATTCAGCCAGATCGGCATC-3′; | (SEQ ID NO: 10) |

and full-length complements thereof.

39. A diagnostic kit for detecting a metallo-beta-lactamase nucleic acid, wherein the kit comprises:
(a) at least one primer pair capable of hybridizing to a beta-lactamase nucleic acid;
(b) at least one positive control and at least one negative control; and
(c) a protocol for identification of the beta-lactamase nucleic acid, wherein the primers are selected from the group consisting of:

| | |
|---|---|
| 5′-GGAATAGAGTGGCTTAATTC-3′; | (SEQ ID NO: 1) |
| 5′-CAACCAGTTTTGCCTTACC-3′; | (SEQ ID NO: 2) |
| 5′-CCTCACATTTCCATAGCGAC-3′; | (SEQ ID NO: 3) |
| 5′-GTAAGCTTCAAGAGCGACG-3′; | (SEQ ID NO: 4) |
| 5′-CGAGAAGCTTGAAGAAGGT-3′; | (SEQ ID NO: 5) |
| 5′-GCTGTCGCTATGGAAATGTG-3′; | (SEQ ID NO: 6) |
| 5′-GGTGTAGTCACAAAACACGG-3′; | (SEQ ID NO: 7) |
| 5′-CAGGTAACCAAACCACTACG-3′; | (SEQ ID NO: 8) |
| 5′-GGTGTTTGGTCGCATATCGC-3′; | (SEQ ID NO: 9) |
| 5′-CCATTCAGCCAGATCGGCATC-3′; | (SEQ ID NO: 10) |

and full-length complements thereof.

40. The method of claim 6 wherein the primers are selected from the group consisting of:

| | |
|---|---|
| 5′-GGAATAGAGTGGCTTAATTC-3′; | (SEQ ID NO: 1) |
| 5′-CAACCAGTTTTGCCTTACC-3′; | (SEQ ID NO: 2) |
| 5′-CCTCACATTTCCATAGCGAC-3′; | (SEQ ID NO: 3) |
| 5′-GTAAGCTTCAAGAGCGACG-3′; | (SEQ ID NO: 4) |
| 5′-CGAGAAGCTTGAAGAAGGT-3′; | (SEQ ID NO: 5) |
| 5′-GCTGTCGCTATGGAAATGTG-3′; | (SEQ ID NO: 6) |
| 5′-GGTGTAGTCACAAAACACGG-3′; | (SEQ ID NO: 7) |
| 5′-CAGGTAACCAAACCACTACG-3′; | (SEQ ID NO: 8 |

and full-length complements thereof.

41. The method of claim 6 wherein the nucleic acid is characteristic of an IMP-1, IMP-4, IMP-5, IMP-6, IMP-7, IMP-10, or IMP-18 beta-lactamase enzyme.

42. The method of claim 6 wherein the nucleic acid is characteristic of an IMP-11 or IMP-21 beta-lactamase enzyme.

43. The method of claim 6 wherein the nucleic acid is characteristic of an IMP-2, IMP-8, IMP-13, or IMP-19 beta-lactamase enzyme.

44. The method of claim 43 wherein the primers are selected from the group consisting of:

| | |
|---|---|
| 5′-CGAGAAGCTTGAAGAAGGT-3′; | (SEQ ID NO: 5) |
| 5′-GCTGTCGCTATGGAAATGTG-3′; | (SEQ ID NO: 6) |

and full-length complements thereof.

45. The method of claim 6 wherein the nucleic acid is characteristic of an IMP-9 beta-lactamase enzyme.

46. The method of claim 45 wherein the primers are selected from the group consisting of:

| | |
|---|---|
| 5′-GGAATAGAGTGGCTTAATTC-3′; | (SEQ ID NO: 1) |
| 5′-CAACCAGTTTTGCCTTACC-3′; | (SEQ ID NO: 2) |
| 5′-CCTCACATTTCCATAGCGAC-3′; | (SEQ ID NO: 3) |
| 5′-GTAAGCTTCAAGAGCGACG-3′; | (SEQ ID NO: 4) |

and full-length complements thereof.

47. The method of claim 6 wherein the nucleic acid is characteristic of an IMP-18 beta-lactamase enzyme.

48. The method of claim 47 wherein the primers are selected from the group consisting of:

| | |
|---|---|
| 5′-GGAATAGAGTGGCTTAATTC-3′; | (SEQ ID NO: 1) |
| 5′-CAACCAGTTTTGCCTTACC-3′; | (SEQ ID NO: 2) |
| 5′-GGTGTAGTCACAAAACACGG-3′; | (SEQ ID NO: 7) |
| 5′-CAGGTAACCAAACCACTACG-3′; | (SEQ ID NO: 8 |

and full-length complements thereof.


SEQUENCE LISTING (10 sequences)

SEQ ID NO: 1 - DNA, 20 bp, ARTIFICIAL
  Primer
ggaatagagtggcttaattc

SEQ ID NO: 2 - DNA, 19 bp, ARTIFICIAL
  Primer
caaccagttttgccttacc

SEQ ID NO: 3 - DNA, 20 bp, ARTIFICIAL
  Primer
cctcacatttccatagcgac

SEQ ID NO: 4 - DNA, 19 bp, ARTIFICIAL
  Primer
gtaagcttcaagagcgacg

SEQ ID NO: 5 - DNA, 19 bp, ARTIFICIAL
  Primer
cgagaagcttgaagaaggt

SEQ ID NO: 6 - DNA, 20 bp, ARTIFICIAL
  Primer
gctgtcgctatggaaatgtg

SEQ ID NO: 7 - DNA, 20 bp, ARTIFICIAL
  Primer
ggtgtagtcacaaaacacgg

SEQ ID NO: 8 - DNA, 20 bp, ARTIFICIAL
  Primer
caggtaaccaaaccactacg

SEQ ID NO: 9 - DNA, 20 bp, ARTIFICIAL
  Primer
ggtgtttggtcgcatatcgc

SEQ ID NO: 10 - DNA, 21 bp, ARTIFICIAL
  Primer
ccattcagccagatcggcatc